(19) United States
(12) Patent Application Publication
(10) Pub. No.: US 2008/0293148 A1
(43) Pub. Date: Nov. 27, 2008

(54) Method and System for Detecting Bio-Element

(75) Inventors: Tae Song Kim, Seoul (KR); Dae Sung Yoon, Gyeonggi-do (KR); Kyo Seon Hwang, Gyeonggi-do (KR); Jeong Hoon Lee, Seoul (KR)

Correspondence Address: FENWICK & WEST LLP, SILICON VALLEY CENTER, 801 CALIFORNIA STREET, MOUNTAIN VIEW, CA, 94041, US

(73) Assignee: KOREAN INSTITUTE OF SCIENCE AND TECHNOLOGY, Seoul (KR)

(21) Appl. No.: 11/662,914

(22) PCT Filed: Sep. 15, 2005
(86) PCT No.: PCT/KR05/03066

§ 371 Date: Mar. 14, 2007

(30) Foreign Application Priority Data
Sep. 16, 2004 (KR) 10-2004-0074116

(51) Int. Cl. G01N 33/00; B01J 19/00; G01N 21/00

(52) U.S. Cl. 436/86; 436/94; 422/68.1; 422/82.05

(57) ABSTRACT

Disclosed are system and method for measuring a bio-element capable of accurately detecting whether a bio-element such as protein, gene and the like is present in an atmosphere or vapor phase having controlled temperature and humidity thereof and measuring a content of the bio-element. According to an embodiment of the invention, the method comprises steps of preparing a cantilever sensor having a plurality of cantilevers; measuring a basis resonant frequency for the plurality of cantilevers; reacting the cantilevers with a sample including a bio-element; measuring resonant frequencies of the cantilevers after the reaction, in a closed system that is isolated from an exterior environment and temperature and humidity thereof are controlled to a specific state; and calculating variations of the resonant frequencies of the cantilevers before and after the reaction to carry out a quantitative analysis of the bio-element included in the sample.

### TECHNICAL FIELD

The present invention relates to system and method for measuring a bio-element, and more particularly to system and method for measuring a bio-element capable of accurately detecting whether a bio-element such as protein, gene and the like is present in an atmosphere or vapor phase having controlled temperature and humidity thereof and measuring a content of the bio-element.

### BACKGROUND ART

In recent years, there have been actively performed researches for developing a cantilever based sensor so as to detect a physical phenomenon or chemical reaction, which sensor is manufactured through a micro electro mechanical system (MEMS) process.

The cantilever based sensors that are currently researched adopt a manner of measuring a mass change with a light source such as laser, which change results from heat or gas adsorption in the atmosphere or liquid. In other words, most of cantilever based sensors uses an optic system to measure a static deflection due to a surface change of the cantilever.

An example of applying the sensing method by the deflection to detection of a bio-element is introduced in Nature Biotechnology 19, 856-860 (2001) and Science 288, 316-318 (2000), which discloses a method of detecting protein and gene through a biological reaction occurring on a surface of a micro cantilever. The sensing methods by the static deflection are carried out in a manner of illuminating a surface of the cantilever with a light source such as laser on and focusing the light with a position sensing diode to detect whether or not the protein or gene. However, the detection of the biological reaction in the system measuring a displacement of the cantilever is mainly performed in the liquid. In the optical measurement method of the cantilever in the liquid, there exist experimental errors such as signal attenuation, parasitic deflection, narrow dynamic range and the like due to the measurement in the liquid.

In the mean time, in addition to the optical measurement method for the cantilever displacement, there has been progressed a research on a micro cantilever sensor using a frequency change. Thundat et al. reported that it was possible to measure changes of spring constant by adsorption of Na⁺ ions on a surface of a micro cantilever sensor through a resonant frequency measurement (Applied Physics Letters 80, 2219-2221 (2002)). Also, the IBM research institute in Zurich, Swiss and some researchers reported that it was possible to detect specific gases in the air through the resonant frequency measurement. For example, U.S. Pat. No. 5,719,324 discloses a cantilever sensor using a reaction of chemical materials on a cantilever, wherein a variation of resonant frequency is used to analyze a target chemical material. In addition, U.S. Pat. Nos. 6,212,939 and 6,289,717 disclose a chemical sensor using adsorption on a silicon cantilever and a sensor combining a binding partner of a material which is desired to detect on a cantilever and then detecting the material.

However, since the sensors disclosed in the U.S. Patents detect the reaction in the liquid phase and the variations of the displacement or resonant frequency of the cantilever in the liquid using an optic method or detect the variation of resonant frequency through an exterior oscillator, they have the problems due to the measurement in the liquid as described above. In addition, the prior art is limited to a chemical material of vapor phase (U.S. Pat. No. 5,719,324) or focuses on the resonance using an external piezoelectric material or the sensing method using optics. Although U.S. Pat. No. 6,289,717 mentions a micro mechanical antibody sensor, it is limited to the measurement of the cantilever displacement by an optical method in the liquid, rather than an electric measurement method using a cantilever. Additionally, it is suggested a method of detecting prostate specific antigen (PSA; indicator protein of prostatic cancer) by a bending method in Nature Biotechnology, 19 pp 856-860. This suggestion is also a research on a liquid phase reaction of target molecules and an optical measurement method.

As described above, the cantilever sensors according to the prior art can measure the adsorption of liquid or vapor phase and biological reaction. However, the electric measurement in the liquid rather than optical measurement has large experimental errors due to the variations of resonant frequency as density and viscosity of the liquid are varied, and should endure low sensitivity due to damping. In addition, the measurement for antigen-antibody reaction using the prior cantilever sensor is mainly performed at room temperature or less (about 28° C.).

### DISCLOSURE OF INVENTION

### Technical Problem

Accordingly, the present invention has been made to solve the above problems. An object of the invention is to provide system and method for measuring a bio-element capable of accurately detecting whether a bio-element such as protein, gene and the like is present in an atmosphere or vapor phase having controlled temperature and humidity thereof and measuring a content of the bio-element.

### Technical Solution

In order to achieve the above object, there is provided a bio-element measuring method comprising steps of: preparing a cantilever sensor having a plurality of cantilevers; measuring a basis resonant frequency for the plurality of cantilevers; reacting the cantilevers with a sample including a bio-element; measuring resonant frequencies of the cantilevers after the reaction, in a closed system that is isolated from an exterior environment and temperature and humidity thereof are controlled to a specific state; and calculating variations of the resonant frequencies of the cantilevers before and after the reaction to carry out a quantitative analysis of the bio-element included in the sample.

Preferably, the method of the invention may further comprising a step of cleaning and drying the cantilevers using one of ultra pure water and buffer solution before measuring the resonant frequencies of the cantilevers after the reaction with the sample.

Preferably, at least one of the cantilevers may have a molecule recognition layer on at least one of upper and lower surfaces of the cantilever.

Preferably, at least one of the cantilevers may don't have a molecule recognition layer on at least one of upper and lower surfaces of the cantilever.

Preferably, the molecule recognition layer may comprise a monoatomic layer.

Preferably, the temperature of the closed system may be 10˜60° C.

Preferably, the temperature of the closed system may be 30˜40° C.

Preferably, the humidity of the closed system may be a relative humidity of 10˜90%.

Preferably, the humidity of the closed system may be a relative humidity of 70˜90%.

Preferably, the plurality of cantilevers may be integrated with a piezoelectric film.

Preferably, the plurality of cantilevers may be integrated with a piezoresistive film.

According to another embodiment of the invention, there is provided a bio-element measuring method comprising steps of: preparing a cantilever sensor having a plurality of cantilevers; measuring a basis displacement value for the plurality of cantilevers using optic means; reacting the cantilevers with a sample including a bio-element; measuring displacement values of the cantilevers after the reaction, in a closed system that is isolated from an exterior environment and temperature and humidity thereof are controlled to a specific state; and calculating variations of displacement values of the cantilevers before and after the reaction to carry out a quantitative analysis of the bio-element included in the sample.

According to another aspect of the invention, there is provided a bio-element measuring system comprising: a closed system defining a predetermined space and isolated from an exterior environment; a sample supply system provided in the closed system and supplying and discharging a sample including a bio-element; a reaction chamber provided in the closed system, connected to the sample supply system to provide a space capable of receiving the sample and mounted with a cantilever sensor having a plurality of cantilevers; temperature control means for controlling a temperature of the closed system; and humidity control means for controlling humidity of the closed system.

Preferably, at least one of the cantilevers may have a molecule recognition layer on at least one of upper and lower surfaces of the cantilever.

Preferably, at least one of the cantilevers may don't have a molecule recognition layer on at least one of upper and lower surfaces of the cantilever.

Preferably, the molecule recognition layer may comprise a monoatomic layer.

Preferably, the temperature in the closed system may be controlled in a temperature range of 10˜60° C., more preferably 30˜40° C.

Preferably, the humidity of the closed system may be controlled in a relative humidity range of 10˜90%, more preferably 70˜90%.

Preferably, the plurality of cantilevers may be integrated with a piezoelectric film.

Preferably, the bio-element measuring system may further comprise a power supply section and resonant frequency measuring means for measuring the resonant frequencies for the cantilevers.

Preferably, the bio-element measuring system may further comprise optic means for measuring displacement values for the cantilevers.

Preferably, the humidity control means may comprise storage means having a volume in which liquid for controlling humidity is stored.

Preferably, the liquid for controlling humidity may be one of ultra pure water and buffer solution.

Preferably, the sample supply system may comprise a sample inlet through which the sample flows in, a sample pipe connected to the reaction chamber and supplying the sample to the reaction chamber, and a sample outlet provided to a side of the reaction chamber and discharging the sample in the reaction chamber.

According to the invention, it is measured the basis resonant frequency or basis displacement value for the cantilevers before the cantilevers react with the sample including the bio-element to be measured. After the reaction with the sample, it is measured the resonant frequencies or displacement values in the closed system having temperature and humidity controlled to the specific state. Accordingly, it is possible to carry out a quantitative analysis of the bio-element in the sample through variations of the resonant frequencies before and after the reaction. Since the system and method of the invention adopt a measurement manner in the atmosphere or air, rather than in the liquid as the prior art, it is possible to eliminate the problems resulting from the measurement in the liquid and thus to perform accurate quantitative and qualitative analyses.

### ADVANTAGEOUS EFFECTS

System and method for measuring bio-element according to the invention have following effects. According to the invention, it is measured the basis resonant frequency or basis displacement for the cantilever before the reaction with the sample having the bio-element to be measured included therein. After the reaction, it is measured resonant frequency or displacement for the cantilever in the closed system having temperature and humidity controlled to specific states. Through the measures, it is possible to perform the quantitative analysis of the bio-element included in the sample from the variations of the resonant frequency or displacement before and after the reaction.

In particular, according to the invention, since the measurements are carried out in the atmosphere or air, rather than in the liquid as the prior art, it is possible to solve the problems due to the measurements in the liquid, for example, the experimental errors such as the variations of the resonant frequency by the ostensible reasons such as viscosity or density of the liquid.

In addition, according to the invention, it is possible to solve the experimental errors and the sensitivity deterioration due to the parasitic deflection and narrow dynamic range which are problems of the displacement measurement method using the optic system according to the prior art.

### BRIEF DESCRIPTION OF THE DRAWINGS

FIG. 1 is a perspective view of a bio-element measuring system according to an embodiment of the invention;

FIG. 2 is a sectional view taken along a line A-A′ in FIG. 1;

FIG. 3 is an enlarged perspective view of a cantilever sensor shown in FIG. 1;

FIG. 4 is a graph showing variations of resonant frequency before and after a reaction as a temperature of a cantilever having no monoatomic layer is varied, under state that a relative humidity is fixed to be 60%;

FIG. 5 is a graph showing variations of resonant frequencies before and after a reaction as temperatures of a cantilever (A) having a monoatomic layer only and a cantilever (B) having a bio-element trapped in a monoatomic layer through the reaction are varied, under state that a relative humidity of a closed system is fixed to be 60%;

FIG. 6 is a graph showing variations of resonant frequencies of cantilevers as a relative humidity is varied, under state that a temperature of a closed system is fixed to be 37° C.;

FIG. 7 is a graph showing variations of resonant frequencies of cantilevers, under state that a temperature and a relative humidity of a closed system are fixed to be 37° C. and 80%, respectively; and

FIG. 8 is a flow chart for illustrating a bio-element measuring method according to an embodiment of the invention.

### DESCRIPTION OF MAIN PARTS OF THE DRAWINGS

101: Reaction chamber 110: Substrate
120: Cantilever 121: Upper electrode
122: Piezoelectric film 123: Lower electrode
124: Buffer film 125: Supper film
126: Detection film 127: Monoatomic layer
131: Sample inlet 132: Sample pipe
133: Sample outlet 134: Sample regulating valve
141: Storage means 150: Temperature control means
160: Means for measuring a resonant frequency
170: Power supply section

### BEST MODE FOR CARRYING OUT THE INVENTION

Hereinafter, a preferred embodiment of the present invention will be described with reference to the accompanying drawings. In the following description of the present invention, a detailed description of known functions and configurations incorporated herein will be omitted when it may make the subject matter of the present invention rather unclear.

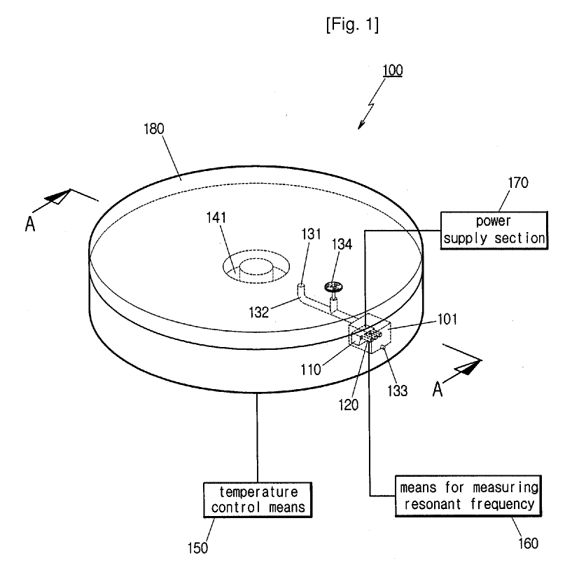

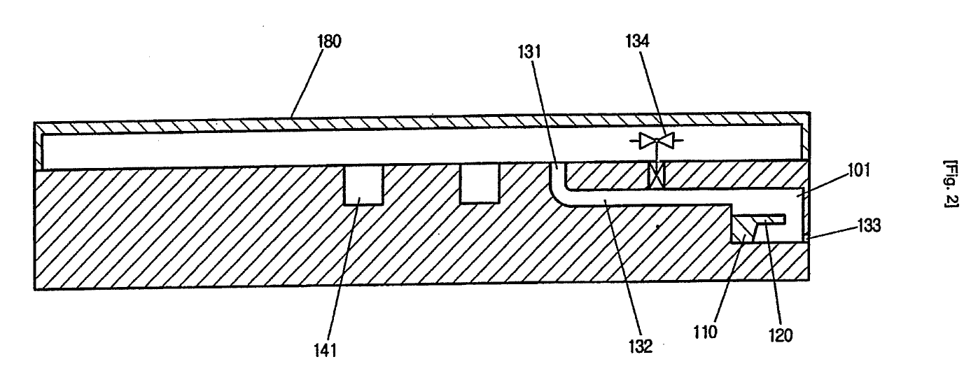

FIG. 1 is a perspective view of a bio-element measuring system according to an embodiment of the invention, and FIG. 2 is a sectional view taken along a line A-A′ in FIG. 1.

A core of the technical spirit of system and method of measuring a bio-element according to the invention is that the bio-element is measured in the atmosphere or vapor state in a space having controlled temperature and humidity. It should be noted that FIGS. 1 and 2 are example for embodying the system and method of measuring a bio-element according to the invention and a variety of modifications thereof can be made.

Referring to FIGS. 1 and 2, a bio-element measuring system 100 is generally divided into a sample supply system and a reaction chamber 101. The sample supply system supplies a liquid sample including a bio-element to be measured, for example blood and the like to the reaction chamber 101, and comprises a sample inlet 131, a sample pipe 132 and a sample outlet 133. A sample regulating valve 134 is provided to a side of the sample pipe 132 to regulate supply and interruption of the sample. The sample outlet 133 is provided to a side of the reaction chamber 101 to discharge the sample having been completely reacted. Although it is not shown in FIG. 1, a sample discharge valve is provided to an end of the sample outlet 133 to regulate the sample discharge. In the mean time, the sample supply system also serves as a cleaning liquid supply system. In other words, when a reaction is completed after the sample is supplied to the reaction chamber, which will be specifically described later, it is possible to supply and discharge cleaning liquid for cleaning a cantilever through the sample inlet, the sample pipe and the sample outlet of the sample supply system.

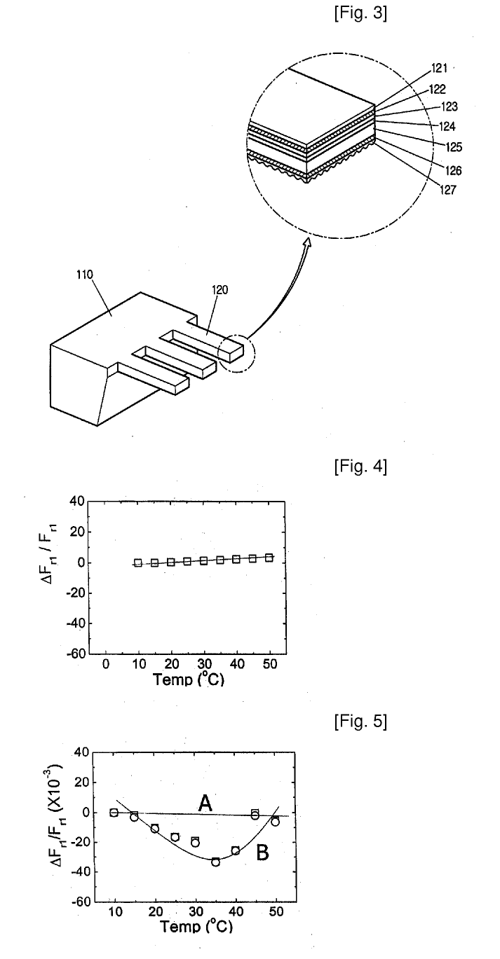

The reaction chamber 101 has a predetermined reaction space for receiving the sample and a cantilever sensor is mounted in the reaction chamber 101. The cantilever sensor comprises a substrate 110 and at least one cantilever 120. The cantilever 120 is meant that it comprises a cantilever having a minimum structure. That is, the cantilever 120 applied to the invention comprises all cantilevers having a molecule recognition layer serving to trap a bio-element. For example, it is comprised all cantilevers having a molecule recognition layer which is a minimum structure condition as means for detecting a bio-element, such as cantilever having a separate piezoelectric transducer in U.S. Pat. No. 5,719,324 and cantilever having integrated a piezoelectric film or piezoresistive film in Korean Patent Application No. 2003-92618 that was filed by the applicant. The molecule recognition layer is formed on at least one of upper and lower surfaces of the cantilever 120 and comprises a detection film 126 made of a conductive material such as gold (Au) and a monoatomic layer 127 formed on the detection film 126, as shown in FIG. 3. The molecule recognition layer is sometimes meant by the detection film only or the monoatomic layer only. The monoatomic layer is also referred to as a self assembled monolayer (SAM) and serves to substantially trap the bio-element to be measured which is included in the sample. In addition, at least one of the plurality of cantilevers is not provided with the molecule recognition layer. The reason not to provide the molecule recognition layer to at least one of the cantilevers is to correct an error occurring when the bio-element trapped in the molecule recognition layer is not a bio-element to be measured. This will be specifically described hereinafter.

### MODE FOR THE INVENTION

The cantilever sensor shown in FIG. 3 is a cantilever sensor having a piezoelectric film integrated thereto as disclosed in Korean Patent Application No. 2003-92618 that was filed by the applicant, and has such structure that an upper electrode 121, a piezoelectric film 122, a lower electrode 123, a buffer film 124, a support film 125, a detection film 126 and a monoatomic layer 127 are sequentially stacked. Hereinafter, the cantilever sensor having the piezoelectric film 122 integrated thereto will be described.

In addition to the sample supply system 131, 132, 133 and the reaction chamber 101, the bio-element measuring system of the invention further comprises a cap 180 having a predetermined shape and isolating the sample supply system and the reaction chamber from the exterior environment. The bio-element measuring system 100 of the invention forms a closed system due to the cap 180. Meanwhile, the bio-element measuring system 100 comprises humidity control means and temperature control means 150. The humidity control means is means for controlling humidity of the closed system in the cap 180 and comprises storage means 141 at a side in the cap in which liquid for controlling humidity, for example ultra pure water (deionized water) or phosphate buffered solution (PBS) can be stored. An amount of the liquid for controlling humidity stored in the storage means 141 can be properly regulated depending on desired humidity. The temperature control means 150 serves to control a temperature of the closed system defined by the cap.

In the mean time, the cantilever sensor provided in the reaction chamber 101 may be further provided with a power supply section 170 supplying power to the cantilever 120 and means 160 for measuring a resonant frequency delivered from the cantilever 120. In addition, in case of measuring a displacement of a static deflection of the cantilever sensor, although it is not shown, an optic system comprising a laser diode for measuring the displacement may be provided to the bio-element measuring system.

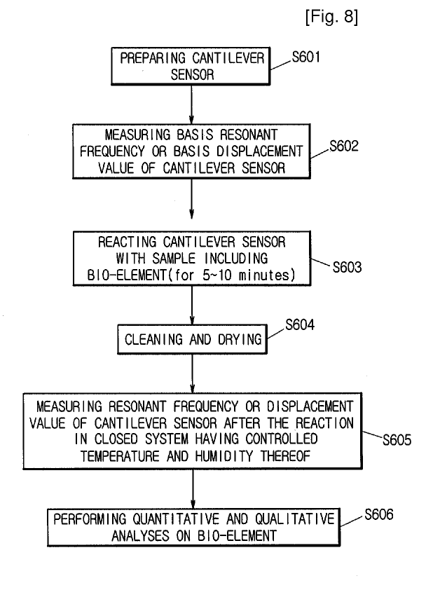

Hereinafter, a bio-element measuring method according to the invention will be specifically described with reference to FIG. 8. As described above, the bio-element measuring method according to the invention is not limited to embodiments using the bio-element measuring system shown in FIGS. 1 and 2. In other words, the method of invention can be embodied using a variety of modified embodiments in addition to the bio-element measuring system shown in FIGS. 1 and 2.

First, according to the bio-element measuring method of the invention, a cantilever sensor is prepared (S601). The cantilever sensor is meant by a cantilever sensor comprising a molecule recognition layer that is the minimum structure element of the cantilever sensor for trapping a bio-element and includes all cantilever sensors having the molecule recognition layer. The molecule recognition layer comprises a detection film made of a conductive material and a monoatomic layer formed on the detection film and is sometimes meant by the detection film only or the monoatomic layer only, as described above. In addition, the cantilever sensor comprises a substrate and a plurality of cantilevers. At least one of the cantilevers may not be provided with the molecule recognition layer. A shape of the cantilever can be modified into a variety of forms depending on uses thereof. The cantilever shown in FIGS. 1 and 3 has a rectangular parallelepiped shape. For example, the cantilever has 1˜400 □ of length and width and 0.1˜10 □ of thickness. The dimensions may be also variously modified. In addition, the cantilever may consist of single crystal silicon, silicon nitride film (SiNₓ), carbon crystals and the like.

When the cantilever sensor is prepared, an electric field is applied to the cantilever sensor to measure a basis resonant frequency of the cantilever (S602). At this time, the basis resonant frequency is measured to compare it with a resonant frequency after a reaction.

In the mean time, at least one of the cantilevers is subject to the resonant frequency measurement, under state that the molecule recognition layer is not provided to it. The reason to measure the resonant frequency for the cantilever having no molecule recognition layer is as follows. The essential characteristics of the bio-element measuring method according to the invention is to measure a basis resonant frequency for a cantilever having a molecule recognition layer and again to measure a resonant frequency for the corresponding cantilever after a reaction, thereby recognizing a bio-element through a variation of the resonant frequencies before and after the reaction. However, since a target sample also includes other bio-elements besides the bio-element to be measured, the other bio-elements can be trapped to the molecule recognition layer after the reaction. In this case, with regard to whether or not the correct bio-element and a content of the element, there may occur an error. In order to prevent the problem, a cantilever having no molecule recognition layer is provided. By providing the cantilever having no molecule recognition layer, it is possible to prevent a non-specific binding and to minimize a room for an error occurrence from a measurement environment.

When the bio-element measuring system shown in FIGS. 1 and 2 is used, the process of measuring the basis resonant frequency can be carried out as follows: the electric field is applied to the cantilever sensor from the power supply section 170, an electric signal is converted into a mechanical vibration through the piezoelectric film 122 provided to the cantilever sensor and the mechanical vibration is again converted into the electric signal, so that a resonant frequency of a corresponding cantilever is finally measured through the resonant frequency measuring means 160.

In the mean time, as described above, the method of measuring a basis resonant frequency has been suggested to set a basis value before the reaction. However, it can be applied a method of measuring a displacement by static deflection of a cantilever as the basis value, in addition to the basis resonant frequency. In other words, displacements of a cantilever before and after the reaction are optically measured through a laser diode and the like to determine whether or not a bio-element trapped in the corresponding cantilever, and a content of the bio-element. At this time, the process of measuring the displacement before the reaction can be referred to as a process of measuring a basis displacement (S602). In addition, similarly to the measurement of the basis resonant frequency, the process of measuring a basis displacement is carried out under state that at least one of the cantilevers is not provided with the molecule recognition layer. The reason not to provide the molecule recognition layer to the cantilever is same as the case of the process of measuring the basis resonant frequency.

When the process of measuring the basis resonant frequency or basis displacement is completed, a reaction process proceeds (S603). The reaction process is meant that a sample including a bio-element to be measured, for example blood is reacted with the cantilever sensor. An example of the reaction process will be described with reference to the bio-element measuring system shown in FIGS. 1 and 2.

The sample such as blood is poured into the sample inlet 131 and flows in the reaction chamber 101 having the cantilever sensor via the sample pipe 132. Under state that the sample has flowed in the reaction chamber 101, a reaction is progressed for about 5˜100 minutes. Through the reaction, a bio-element to be measured, for example prostate specific antigen (PSA) which is an indicator protein of prostatic cancer is trapped on a surface of the molecule recognition layer, specifically monoatomic layer 127 (in FIG. 3) provided to the cantilevers 120. In the reaction process, a substrate such as bovine serum albumin (BSA) may be added to prevent a non-specific binding when fixing the antigen to the molecule recognition layer of the cantilever. When the reaction is completed, the sample having been completely reacted is discharged through the sample outlet.

Under state that the reaction has been completed, the cantilever sensor is cleaned and dried (S604). The cleaning process may be carried out using a buffer solution such as phosphate buffered solution (PBS). When the bio-element measuring system shown in FIGS. 1 and 2 is used, the buffer solution such as PBS is poured into the sample inlet to clean the cantilever sensor in the reaction chamber. When the cleaning process has been completed, a drying process is progressed. At this time, a spin dry method of rotating the bio-element measuring system may be used to perform the drying process.

Under state that the washing and drying processes have been completed, a process of measuring a bio-element is progressed in earnest. The process of measuring a bio-element is performed in the closed system having controlled temperature and humidity thereof to a specific state (S605, S606). The closed system is meant that the cantilever sensor having completed the reaction is isolated from an exterior environment. The bio-element measuring system shown in FIG. 1 may be used as an example of the closed system. Hereinafter, the process of measuring a bio-element using the system shown in FIG. 1 will be described.

First, the reason to fix the temperature and humidity in the closed system to a specific state is to optimize the determination of whether or not the bio-element trapped in the cantilever sensor and a content of the bio-element. For doing so, the temperature and humidity should be able to be controlled. The temperature control means 150 and the humidity control means serve to control the temperature and humidity, respectively. The temperature control means may comprise a heating block and a control device thereof. The humidity control means is provided with the storage means 141 in which the liquid for controlling humidity, for example ultra pure water (deionized water) or PBS is stored to properly control the humidity in the closed system, as described above with regard to FIG. 1.

In order to find an optimal measurement condition, and the content of the bio-element, the temperatures and humidity were variously applied in experiments relating to the invention. Specifically, it was checked behaviors of the resonant frequency change as the temperature was varied under fixed humidity conditions and as the humidity was varied under fixed temperature conditions. The specific conditions of the temperature and humidity were 10˜60° C. and 10˜90% relative humidity.

FIGS. 4 and 5 are graphs showing the variations of the resonant frequency as the temperature is varied under state that the relative humidity is fixed to be 60%. The variation of the resonant frequency is meant by a difference between the basis resonant frequency before the reaction and the resonant frequency of the cantilever after the reaction. Specifically, FIG. 4 is a graph showing the variations of resonant frequency of a cantilever having no molecule recognition layer, and FIG. 5 is a graph showing variations of resonant frequencies for a cantilever (A) having a monoatomic layer only and a cantilever (B) having a bio-element trapped in a monoatomic layer through the reaction.

As shown in FIG. 4, the cantilever having no monoatomic layer, i.e., cantilever having gold (Au) layer only as the detection film exhibited 1˜10 Hz/° C. of variation of resonant frequency as the temperature increased. The reason that variation of resonant frequency occurred despite having no monoatomic layer is due to a coefficient of thermal expansion and moisture adsorption of the cantilever consisting of a plurality of films.

In FIG. 5, the cantilever (A) having the monoatomic layer only on the detection film exhibited a minute variation of resonant frequency as the temperature was varied, similarly to the graph in FIG. 4. To the contrary, the cantilever (B) having the bio-element trapped in the monoatomic layer exhibited a large variation of resonant frequency as the temperature was varied. In particular, the variation of resonant frequency was maximized in the temperature range of 30˜40° C. The maximization of the variation of resonant frequency in specific temperature range means that the measurement of content of the bio-element trapped in the cantilever is optimized in the corresponding temperature range. In other words, it can be seen that the temperature range of 30˜40° C. is an optimal measurement condition when measuring the content of the bio-element trapped in the cantilever. It is presumed that the result (B) shown in FIG. 5 is related to an activity and a reactivity of the bio-element trapped in the monoatomic layer of the cantilever. A bio-element having higher activity has a characteristic that the variation of resonant frequency thereof is larger as the temperature increases.

In the mean time, in the graph (B) of FIG. 5, the variation of resonant frequency is increased in the temperatures of 45° C. or more. It is presumed that such feature results from deterioration of characteristic of the bio-element, for example indicator protein of prostatic cancer (PSA). Through observation of the results obtained as the temperature increases or decreases, it is possible to carry out an application for the characteristic deterioration and structure change of the bio-element using the feature. In other words, it is possible to analyze characteristics of the bio-element through observation of a hysteresis curve in which a reversible reaction occurs.

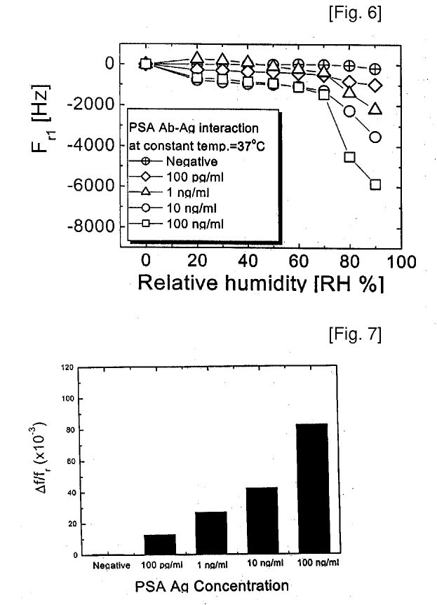

Under state that the optimal temperature condition for the cantilever measurement is determined from the graph in FIG. 5, it is required to determine an optimal humidity condition in the closed system. FIG. 6 shows variations of resonant frequency as the relative humidity is varied, under state that the temperature in the closed system is fixed to be 37° C. belonging to the temperature range of 30˜40° C. In addition, FIG. 6 shows a graph of experimental values for five samples wherein variations of resonant frequency are shown for four samples in which the indicator protein of prostatic cancer is included in concentrations of 100 pg/□, 1 ng/□, 10 ng/□ and 100 ng/□. For reference, the negative graph in FIG. 6 shows a variation of resonant frequency for a cantilever having no monoatomic layer. As shown in FIG. 6, the cantilever (negative) having no monoatomic layer exhibited a substantial constant variation of resonant frequency irrespective of the changes of humidity. To the contrary, as the concentration of the indicator protein of prostatic cancer (PSA) is increased, the variation of the resonant frequency is linearly decreased. This feature results from a force between the proteins, i.e., an electrostatic force or attractive force between molecules (steric force). From the graph in FIG. 6, it can be seen that an optimal humidity condition is 70% or more.

In the mean time, the result as shown in FIG. 6 provides three important information. Specifically, the result provides information about activity and characteristic of a bio-element. Secondly, quantitative analysis materials as a bio-sensor are provided by analyzing a behavior of a bio-element with regard to the humidity. In other words, an application to a bio-element chip becomes possible. Thirdly, a measurement method in the atmosphere or air can be suggested, which is capable of excluding signal attenuation or errors due to a damping occurring from a measurement in the liquid.

From FIGS. 5 and 6, it can be seen that optimal temperature and humidity conditions are 30˜40° C. and 70% or more, respectively. FIG. 7 is a graph showing variations of resonant frequency under state that the temperature and relative humidity are fixed to be 37° C. and 80%. For reference, the sample used in FIG. 7 includes the indicator protein of prostatic cancer (PSA). As shown in FIG. 7, as a concentration of the indicator protein of prostatic cancer is increased in the sample, the variation of resonant frequency is correspondingly increased.

Through a series of processes of preparing the cantilevers, measuring the basis resonant frequency, carrying out the reaction, measuring resonant frequencies after the reaction, calculating the variations of resonant frequency before and after the reaction and the like, it is possible to perform a quantitative analysis of the bio-element in the sample. In addition, since it is possible to determine the reaction degree, i.e., behaviors of the bio-element from the variations of relative humidity or temperature under specific temperature or relative humidity conditions, characteristics of the bio-element can be easily determined.

In the mean time, the invention has been described on the basis of the resonant frequency as shown in FIGS. 4 to 7. However, as described above, it is also possible to perform the quantitative analysis of the bio-element in the sample by measuring a basis displacement of static deflection for the cantilever before the reaction using an optic system, measuring a displacement after the reaction and calculating the variations of displacements before and after the reaction. At this time, the displacement measurement is carried out in a closed system having specific temperature and humidity conditions, similarly to the resonant frequency measurement. Optimal temperature and humidity conditions correspond to the conditions of the resonant frequency measurement, i.e., temperatures of 30˜40° C. and humidity of 70% or more.

In addition, although the embodiments of the invention have been described with reference to the indicator protein of prostatic cancer (PSA) as the bio-element, it is possible to detect a variety of bio-elements such as DNA, cell and the like, in addition to the PSA. Additionally, although the embodiments have been described on the basis of the variations of resonant frequency or displacement using the cantilever, it is possible to make progress the above described processes (S601˜S606) by applying a quartz crystal mass balance (QCM) used for the prior art, instead of the cantilever.

### INDUSTRIAL APPLICABILITY

System and method for measuring bio-element according to the invention have following effects. According to the invention, it is measured the basis resonant frequency or basis displacement for the cantilever before the reaction with the sample having the bio-element to be measured included therein. After the reaction, it is measured resonant frequency or displacement for the cantilever in the closed system having temperature and humidity controlled to specific states. Through the measures, it is possible to perform the quantitative analysis of the bio-element included in the sample from the variations of the resonant frequency or displacement before and after the reaction.

1. A bio-element measuring method comprising steps of:
(a) preparing a cantilever sensor having a plurality of cantilevers;
(b) measuring a basis resonant frequency for the plurality of cantilevers;
(c) reacting the cantilevers with a sample including a bio-element;
(d) measuring resonant frequencies of the cantilevers after the reaction, in a closed system that is isolated from an exterior environment and temperature and humidity thereof are controlled to a specific state; and
(e) calculating variations of the resonant frequencies of the cantilevers before and after the reaction to carry out a quantitative analysis of the bio-element included in the sample.

2. The method according to claim 1, further comprising a step of cleaning and drying the cantilevers using one of ultra pure water and buffer solution before measuring the resonant frequencies of the cantilevers after the reaction with the sample.

3. The method according to claim 1, wherein at least one of the cantilevers has a molecule recognition layer on at least one of upper and lower surfaces of the cantilever.

4. The method according to claim 1, wherein at least one of the cantilevers doesn't have a molecule recognition layer on at least one of upper and lower surfaces of the cantilever.

5. The method according to claim 3, wherein the molecule recognition layer comprises a monoatomic layer.

6. The method according to claim 1, wherein the temperature of the closed system is 10˜60° C.

7. The method according to claim 6, wherein the temperature of the closed system is 30˜40° C.

8. The method according to claim 1, wherein the humidity of the closed system is a relative humidity of 10˜90%.

9. The method according to claim 8, wherein the humidity of the closed system is a relative humidity of 70˜90%.

10. The method according to claim 1, wherein the plurality of cantilevers comprises a cantilever having a piezoelectric film or piezoresistive film integrated with it.

11. The method according to claim 1, further comprising a step of repeating the steps of (a) to (e) at least one time to determine a behavior characteristic of the bio-element as the temperature or humidity is varied in a specific relative humidity or temperature.

12. A bio-element measuring method comprising steps of:
(a) preparing a cantilever sensor having a plurality of cantilevers;
(b) measuring a basis displacement value for the plurality of cantilevers using optic means;
(c) reacting the cantilevers with a sample including a bio-element;
(d) measuring displacement values of the cantilevers after the reaction, in a closed system that is isolated from an exterior environment and temperature and humidity thereof are controlled to a specific state; and
(e) calculating variations of displacement values of the cantilevers before and after the reaction to carry out a quantitative analysis of the bio-element included in the sample.

13. The method according to claim 12, further comprising a step of cleaning and drying the cantilevers using one of ultra pure water and buffer solution before measuring the displacement values of the cantilevers after the reaction with the sample.

14. The method according to claim 11, wherein at least one of the cantilevers has a molecule recognition layer on at least one of upper and lower surfaces of the cantilever.

15. The method according to claim 12, wherein at least one of the cantilevers doesn't have a molecule recognition layer on at least one of upper and lower surfaces of the cantilever.

16. The method according to claim 14, wherein the molecule recognition layer comprises a monoatomic layer.

17. The method according to claim 12, wherein the temperature of the closed system is 10˜60° C.

18. The method according to claim 17, wherein the temperature of the closed system is 30˜40° C.

19. The method according to claim 12, wherein the humidity of the closed system is a relative humidity of 10-90%.

20. The method according to claim 19, wherein the humidity of the closed system is a relative humidity of 70-90%.

21. The method according to claim 12, further comprising a step of repeating the steps of (a) to (e) at least one time to determine a behavior characteristic of the bio-element as the temperature or humidity is varied in a specific relative humidity or temperature.

22. The method according to claim 1, wherein a quartz crystal mass balance (QCM) is used instead of the cantilever sensor.

23. A bio-element measuring system comprising:
a closed system defining a predetermined space and isolated from an exterior environment;
a sample supply system provided in the closed system and supplying and discharging a sample including a bio-element;
a reaction chamber provided in the closed system, connected to the sample supply system to provide a space capable of receiving the sample and mounted with a cantilever sensor having a plurality of cantilevers;
temperature control means for controlling a temperature of the closed system; and
humidity control means for controlling humidity of the closed system.

24. The system according to claim 23, wherein at least one of the cantilevers has a molecule recognition layer on at least one of upper and lower surfaces of the cantilever.

25. The system according to claim 23, wherein at least one of the cantilevers doesn't have a molecule recognition layer on at least one of upper and lower surfaces of the cantilever.

26. The system according to claim 24, wherein the molecule recognition layer comprises a monoatomic layer.

27. The system according to claim 23, wherein the temperature in the closed system is controlled in a temperature range of 10˜60° C.

28. The system according to claim 27, wherein the temperature in the closed system is controlled in a temperature range of 30˜40° C.

29. The system according to claim 23, wherein the humidity of the closed system is controlled in a relative humidity range of 10-90%.

30. The system according to claim 23, wherein the humidity of the closed system is controlled in a relative humidity range of 70-90%.

31. The system according to claim 23, wherein the cantilevers comprises a cantilever having a piezoelectric film or piezoresistive film integrated with it.

32. The system according to claim 23, further comprising a power supply section and resonant frequency measuring means for measuring resonant frequencies for the cantilevers.

33. The system according to claim 23, wherein further comprising optic means for measuring displacement values for the cantilevers.

34. The system according to claim 23, wherein the humidity control means comprises storage means having a volume in which liquid for controlling humidity is stored.

35. The system according to claim 34, wherein the liquid for controlling humidity is one of ultra pure water and buffer solution.

36. The system according to claim 23, wherein the sample supply system comprises:
a sample inlet through which the sample flows in,
a sample pipe connected to the reaction chamber and supplying the sample to the reaction chamber, and
a sample outlet provided to a side of the reaction chamber and discharging the sample in the reaction chamber.

37. The system according to claim 23, wherein a quartz crystal mass balance (QCM) is used instead of the cantilever sensor.

38. The method according to claim 4, wherein the molecule recognition layer comprises a monoatomic layer.

39. The method according to claim 15, wherein the molecule recognition layer comprises a monoatomic layer.

40. The method according to claim 12, wherein a quartz crystal mass balance (QCM) is used instead of the cantilever sensor.

41. The system according to claim 25, wherein the molecule recognition layer comprises a monoatomic layer.
